(19) United States
(12) Patent Application Publication
(10) Pub. No.: US 2005/0239745 A1
(43) Pub. Date: Oct. 27, 2005

(54) Novel topical ophthalmic formulations

(75) Inventors: Mark B. Abelson, Andover, MA (US); Paulo J. Gomes, Haverhill, MA (US); Matthew J. Chapin, Haverhill, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Ophthalmic Research Associates, Inc., North Andover, MA (US)

(21) Appl. No.: 11/074,000

(22) Filed: Mar. 3, 2005

Related U.S. Application Data
(60) Provisional application No. 60/549,703, filed on Mar. 3, 2004.

(51) Int. Cl. A61K 31/716; A61K 31/55; A61K 31/192; A61K 31/205

(52) U.S. Cl. 514/57; 514/569; 514/217.05; 514/554

(57) ABSTRACT

The invention features novel topical formulations of antiallergenic agents in formulation with various other agents (such as a tear substitute) that provide a comfortable formulation when instilled in the eye and has enhanced efficacy and duration of action. The invention further features novel methods of treating ocular allergy by topical application of antiallergenic agents in formulation with various other agents (such as a tear substitute), as well as kits for the use of such novel formulations and methods.

### RELATED APPLICATION INFORMATION

This application claims the benefit of priority to Provisional Patent Application 60/549,703, filed Mar. 3, 2004. This application is hereby incorporated by reference in its entirety.

### BACKGROUND OF THE INVENTION

There exists a need for topical ophthalmic pharmaceutical products to effectively treat allergic conjunctivitis, a disorder that is characterized by the clinical signs and symptoms of eye itching, redness, tearing, and swelling, and is estimated to have a prevalence of over 20% in the United States. The signs and symptoms of allergic conjunctivitis can significantly impact the quality of life of patients, from social interactions, productivity at work and school, to the ability to perform visual tasks such as working on a computer or reading.

The mast cell is the primary cell involved in eye allergy, and when stimulated by an allergen (pollen, dust, dander) releases a host of substances that produce the signs and symptoms of allergic conjunctivitis (itching, redness, swelling, and tearing). Histamine is the primary mediator released and stimulates receptors on nerve endings and blood vessels to produce itching and redness. There are two histamine receptors that have been identified on the ocular surface. H1 receptors on nerve endings lead to itching, and H1 and H2 receptors on blood vessels lead to dilation of the blood vessels, leading to redness, and leakage of fluid from the vessels into the surrounding tissue producing swelling. Allergic conjunctivitis may also co-exist with other external ocular conditions and diseases, such as dry eye. This leads to a compromised tear film, which serves to protect the ocular surface from allergens.

Currently available treatments for eye allergy include: drops which can wash allergens off the ocular surface and act as a barrier for the eye (e.g. artificial tears), drugs which block histamine from binding to the histamine receptors (e.g. antihistamines), drugs that block the release of histamine and other substances from the mast cell (e.g. mast cell stabilizers), drugs with multiple modes of action (e.g. antihistamine/mast cell stabilizing agents), and drugs that can actively constrict blood vessels thus reducing redness and swelling (e.g. vasoconstrictors). The criteria which may be considered in evaluating the appropriateness of an agent for a patient include: efficacy at onset of action, duration of action, how well it controls the individual signs and symptoms of allergic conjunctivitis, and comfort of the drop when instilled in the eye. The comfort of an ophthalmic product depends on the active pharmaceutical ingredient itself, as well as the nature of the formulation and the vehicle that makes up the product. For example, antihistamines have been shown to induce decreased tear production and lead to dryness of the ocular surface, making the eye susceptible to irritation by an ophthalmic product.

Ketotifen fumarate is a pharmaceutical agent having antihistamine, mast-cell stabilizing and anti-inflammatory properties. It is currently approved in the United States as 0.025% ketotifen fumarate ophthalmic solution (Zaditor®-Novartis) and in other territories (including Japan and South America) in a 0.05% formulation. In the United States, the 0.025% formulation is indicated for the temporary prevention of itching of the eye due to allergic conjunctivitis and for twice daily dosing. In controlled clinical studies it has been shown to have a duration of action up to 12 hours. It is generally known that the higher concentration formulations of ketotifen fumarate that are available outside the United States (i.e. 0.05%) is not as comfortable when instilled in the eye and produces some stinging/burning on the ocular surface. Because higher concentrations of active agents may offer greater efficacy, a more comfortable formulation with concentrations of ketotifen fumarate greater than 0.025% is desirable.

### SUMMARY OF THE INVENTION

The invention features novel topical ophthalmic formulations of antiallergenic agents in combination with various other agents (such as a tear substitute) that provide a comfortable formulation when instilled in the eye and have enhanced efficacy and duration of action over formulations of antiallergenic agents that are not combined with such other agents. The invention also features novel methods of treating and preventing ocular allergy with these formulations. Further, the invention features kits for the shipping, storage or use of the formulations, as well the practice of the methods. Other features and advantages of the invention will become apparent from the following detailed description and claims.

### DETAILED DESCRIPTION OF THE INVENTION

1. General

The invention is based in part on the discovery that novel topical ophthalmic formulations of ketotifen fumarate at concentrations of 0.05, 0.075 and 0.1% in combination with a tear substitute such as Genteal® Lubricant eye drops exhibit enhanced efficacy and duration of action when compared to a commercially available 0.025% ketotifen fumarate formulation (Zaditor®). The enhanced efficacy and duration of action may be due to synergism of the ketotifen and the tear substitute in providing a longer dwell time of the ketotifen fumarate on the ocular surface. In addition, certain of these novel formulations are more comfortable than Zaditor® when applied to eye surface of a subject, and certain of these novel formulations allow increased concentrations of ketotifen fumarate to be applied to the eye surface of a subject with less discomfort.

2 Definitions

For convenience, before further description of the present invention, certain terms employed in the specification, examples, and appended claims are collected here. These definitions should be read in light of the remainder of the disclosure and understood as by a person of skill in the art.

As used herein, the term “antiallergenic agent” refers to a molecule or composition that treats eye allergy or reduces a symptom of eye allergy. Examples of antiallergenic agents include, but are not limited to, “antihistamines” or drugs which block histamine from binding to the histamine receptors, “mast cell stabilizers” or drugs that block the release of histamine and other substances from the mast cell, “drugs with multiple modes of action” or drugs that are antiallergenic agents having multiple modes of action (e.g. drugs that are antihistamines and mast cell stabilizers, drugs with antihistamine, mast cell stabilizing and anti-inflammatory activity, etc.), and nonsteroidal anti-inflammatory drugs or “NSAIDs.”

The term “aqueous” typically denotes an aqueous composition wherein the carrier is to an extent of >50%, more preferably >75% and in particular >90% by weight water.

The phrase “effective amount” is an art-recognized term, and refers to an amount of an agent that, when incorporated into a pharmaceutical composition of the present invention, produces some desired effect at a reasonable benefit/risk ratio applicable to any medical treatment. In certain embodiments, the term refers to that amount necessary or sufficient to eliminate, reduce or maintain (e.g., prevent the spread of) a symptom of ocular allergy, or prevent or treat ocular allergy. The effective amount may vary depending on such factors as the disease or condition being treated, the particular composition being administered, or the severity of the disease or condition. One of skill in the art may empirically determine the effective amount of a particular agent without necessitating undue experimentation.

The term “ocular allergy” as used herein refers to any allergic disease of the eye. Examples of such ocular allergies include but are not limited to seasonal/perennial allergic conjunctivitis, vernal keratoconjunctivitis, giant papillary conjunctivitis, perennial allergic conjunctivitis and atopic keratoconjunctivitis. The signs and symptoms of ocular allergies include chemosis, eye itching, redness and swelling.

A “patient,” “subject,” or “host” to be treated by the subject method refers to either a human or non-human animal, such as primates, mammals, and vertebrates.

The phrase “pharmaceutically acceptable” is art-recognized and refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and refers to, for example, pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, sunflower oil, sesame oil, olive oil, corn oil and soybean oil; (10) glycols, such as propylene glycol;
(11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salts” is art-recognized, and refers to relatively non-toxic, inorganic and organic acid addition salts of compositions of the present invention or any components thereof, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci., 66:1-19 (1977).

The term “preventing,” when used in relation to a condition, such as ocular allergy is art-recognized, and refers to administration of a composition which reduces the frequency of, or delays the onset of, symptoms of a medical condition in a subject relative to a subject which does not receive the composition.

As used herein, the term “tear substitute” refers to molecules or compositions which lubricate, “wet,” approximate the consistency of endogenous tears, aid in natural tear build-up, or otherwise provide temporary relief of dry eye symptoms and conditions upon ocular administration.

The term “treating” is an art-recognized term which refers to curing as well as ameliorating at least one symptom of any condition or disease.

“Vasoconstrictors” are drugs that actively constrict blood vessels.

3. Pharmaceutical Compositions

The invention features novel pharmaceutical compositions comprising an effective amount of an antiallergenic agent and a tear substitute in a pharmaceutically acceptable carrier for the treatment and prevention of ocular allergy. The antiallergenic agent and tear substitute may act synergistically to provide a longer dwell time of the antiallergenic agent on the ocular surface, thus increasing duration and efficacy of action.

In certain embodiments, the invention features pharmaceutical compositions comprising an effective amount of an antihistamine and a tear substitute. Exemplary antihistamines include, but are not limited to, pheniramine, emedastine difumarate and levocabastine. In other embodiments, the invention features pharmaceutical compositions comprising an effective amount of a mast cell stabilizer and a tear substitute. Exemplary mast cell stabilizers include, but are not limited to, nedocromil, Iodoxamide, cromolyn, and cromolyn sodium.

In still other embodiments, the invention features pharmaceutical compositions comprising an effective amount of a drug with multiple modes of action and a tear substitute. Exemplary drugs with multiple modes of action include, but are not limited to, azelastine, epinastine, olopatadine and ketotifen fumarate.

In certain embodiments, the drug with multiple modes of action is ketotifen fumarate and the effective amount of ketotifen fumarate is in the range of about 0.025 to about 0.1%. In certain embodiments, the effective amount of ketotifen fumarate is in the range of about 0.03% to about 0.1%. In other embodiment, the effective amount of ketotifen fumarate is in the range of about 0.05% to about 0.075%. In other embodiments, the effective amount of ketotifen fumarate is in the range of about 0.03 to about 0.50%. In still other embodiments, the effective amount of ketotifen fumarate is in the range of about 0.031 to about 0.035%, of about 0.036 to about 0.040%, of about 0.041 to about 0.045, of about 0.046 to about 0.05%, of about 0.051 to about 0.055%, of about 0.056 to about 0.060%, of about 0.061 to about 0.065%, of about 0.066 to about 0.070%, of about 0.071 to about 0.075%, of about 0.076 to about 0.080%, of about 0.081 to about 0.085%, of about 0.086 to about 0.090%, of about 0.091 to about 0.095%, or of about 0.096 to about 0.1%.

In still other embodiments, the invention features pharmaceutical compositions comprising an effective amount of a NSAID and a tear substitute. Exemplary drugs NSAIDs include, but are not limited to, diclofenac and ketorolac tromethamine.

A variety of tear substitutes are known in the art and include, but are not limited to: monomeric polyols, such as, glycerol, propylene glycol, and ethylene glycol; polymeric polyols such as polyethylene glycol; cellulose esters such hydroxypropylmethyl cellulose, carboxy methylcellulose sodium and hydroxy propylcellulose; dextrans such as dextran 70; water soluble proteins such as gelatin; vinyl polymers, such as polyvinyl alcohol, polyvinylpyrrolidone, and povidone; and carbomers, such as carbomer 934P, carbomer 941, carbomer 940 and carbomer 974P. Many such tear substitutes are commercially available, which include, but are not limited to cellulose esters such as Bion Tears®, Celluvisc®, Genteal®, OccuCoat®, Refresh®, Teargen II®, Tears Naturale®, Tears Natural II®, Tears Naturale Free®, and TheraTears®; and polyvinyl alcohols such as Akwa Tears®, HypoTears®, Moisture Eyes®, Murine Lubricating®, and Visine Tears®. Tear substitutes may also be comprised of paraffins, such as the commercially available Lacri-Lube® ointments. Other commercially available ointments that are used as tear substitutes include Lubrifresh PM®, Moisture Eyes PM® and Refresh PM®.

In certain embodiments, the tear substitute contains hydroxypropylmethylcellulose. In certain embodiments, the tear substitute is Genteal® lubricating eye drops. GenTeal® (CibaVision—Novartis) is a sterile lubricant eye drop containing hydroxypropyl methylcellulose 3 mg/g and preserved with sodium perborate.

In certain embodiments, the pharmaceutical compositions of the invention may comprise combinations of at least two antiallergenic agents and a tear substitute. For example, the first antiallergenic agent may be selected from the group consisting of: an antihistamine, a mast cell stabilizer, a drug with multiple modes of action and a NSAID and the second antiallergenic agent may also be antiallergenic agent may be selected from the group consisting of: an antihistamine, a mast cell stabilizer, a drug with multiple modes of action and a NSAID.

In other embodiments, the topical formulations of the invention may comprise an antiallergenic agent and a combination of at least two tear substitutes.

The pharmaceutical compositions of the invention described above may additionally comprise other active ingredients, including, but not limited to, and vasoconstrictors. In certain embodiments, the pharmaceutical compositions of the invention may comprise at least one antiallergenic agent, a tear substitute, and an effective amount of a vasoconstrictor. Exemplary vasoconstrictors include, but are not limited to, naphazoline, antolazine, tetrahydozoline and oxymetazoline. Vasoconstrictors may act as decongestants.

The antiallergenic agents and other active ingredients of the pharmaceutical compositions may be in the form of a pharmaceutically acceptable salt.

Preferably, the pharmaceutical compositions according to the present invention will be formulated as solutions, suspensions and other dosage forms for topical administration. Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, or other types of solid or semi-solid compositions.

Any of a variety of carriers may be used in the formulations of the present invention including water, mixtures of water and water-miscible solvents, such as C₁- to C₇-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. The concentration of the carrier is, typically, from 1 to 100000 times the concentration of the active ingredient.

Additional ingredients that may be included in the formulation include tonicity enhancers, preservatives, solubilizers, non-toxic excipients, demulcents, sequestering agents, pH adjusting agents, co-solvents and viscosity building agents.

For the adjustment of the pH, preferably to a physiological pH, buffers may especially be useful. The pH of the present solutions should be maintained within the range of 4.0 to 8.0, more preferably about 4.0 to 6.0, more preferably about 6.5 to 7.8. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Borate buffers are preferred. Generally, buffers will be used in amounts ranging from about 0.05 to 2.5 percent by weight, and preferably, from 0.1 to 1.5 percent.

Tonicity is adjusted if needed typically by tonicity enhancing agents. Such agents may, for example be of ionic and/or non-ionic type. Examples of ionic tonicity enhancers are alkali metal or earth metal halides, such as, for example, CaCl₂, KBr, KCl, LiCl, Nal, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids which is equivalent to a 0.9% solution of sodium chloride or a 2.5% solution of glycerol. An osmolality of about 225 to 400 mOsm/kg is preferred, more preferably 280 to 320 mOsm.

In certain embodiments, the topical formulations additionally comprise a preservative. A preservative may typically be selected from a quaternary ammonium compound such as benzalkonium chloride, benzoxonium chloride or the like. Benzalkonium chloride is better described as: N-benzyl-N—(C₈-C₁₈ alkyl)-N,N-dimethylammonium chloride. Examples of preservatives different from quaternary ammonium salts are alkyl-mercury salts of thiosalicylic acid, such as, for example, thiomersal, phenylmercuric nitrate, phenylmercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal®II or sorbic acid. Preferred preservatives are quaternary ammonium compounds, in particular benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary contaminations during use caused by bacteria and fungi.

In another embodiment, the topical formulations of this invention do not include a preservative. Such formulations would be useful for patients who wear contact lenses, or those who use several topical ophthalmic drops and/or those with an already compromised ocular surface (e.g. dry eye) wherein limiting exposure to a preservative may be more desirable.

The topical formulation may additionally require the presence of a solubilizer, in particular if the active or the inactive ingredients tends to form a suspension or an emulsion. A solubilizer suitable for an above concerned composition is for example selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of an especially preferred solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another preferred solubilizer is selected from tyloxapol and from a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient.

The formulations may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements and is generally in the range of from approximately 0.0001 to approximately 90% by weight.

Other compounds may also be added to the formulations of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

4. Packaging

The formulations of the present invention may be packaged as either a single dose product or a multi-dose product. The single dose product is sterile prior to opening of the package and all of the composition in the package is intended to be consumed in a single application to one or both eyes of a patient. The use of an antimicrobial preservative to maintain the sterility of the composition after the package is opened is generally unnecessary.

Multi-dose products are also sterile prior to opening of the package. However, because the container for the composition may be opened many times before all of the composition in the container is consumed, the multi-dose products must have sufficient antimicrobial activity to ensure that the compositions will not become contaminated by microbes as a result of the repeated opening and handling of the container. The level of antimicrobial activity required for this purpose is well known to those skilled in the art, and is specified in official publications, such as the United States Pharmacopoeia (“USP”) and corresponding publications in other countries. Detailed descriptions of the specifications for preservation of ophthalmic pharmaceutical products against microbial contamination and the procedures for evaluating the preservative efficacy of specific formulations are provided in those publications. In the United States, preservative efficacy standards are generally referred to as the “USP PET” requirements. (The acronym “PET” stands for “preservative efficacy testing.”)

The use of a single dose packaging arrangement eliminates the need for an antimicrobial preservative in the compositions, which is a significant advantage from a medical perspective, because conventional antimicrobial agents utilized to preserve ophthalmic compositions (e.g., benzalkonium chloride) may cause ocular irritation, particularly in patients suffering from dry eye conditions or pre-existing ocular irritation. However, the single dose packaging arrangements currently available, such as small volume plastic vials prepared by means of a process known as “form, fill and seal”, have several disadvantages for manufacturers and consumers. The principal disadvantages of the single dose packaging systems are the much larger quantities of packaging materials required, which is both wasteful and costly, and the inconvenience for the consumer. Also, there is a risk that consumers will not discard the single dose containers following application of one or two drops to the eyes, as they are instructed to do, but instead will save the opened container and any composition remaining therein for later use. This improper use of single dose products creates a risk of microbial contamination of the single dose product and an associated risk of ocular infection if a contaminated composition is applied to the eyes.

While the formulations of this invention are preferably formulated as “ready for use” aqueous solutions, alternative formulations are contemplated within the scope of this invention. Thus, for example, the active ingredients, surfactants, salts, chelating agents, or other components of the ophthalmic solution, or mixtures thereof, can be lyophilized or otherwise provided as a dried powder or tablet ready for dissolution (e.g., in deionized, or distilled) water. Because of the self-preserving nature of the solution, sterile water is not required.

5. Methods of Use

The invention features methods of treating or preventing ocular allergy in a subject comprising use of the novel formulations described above. For example, a method of treating ocular allergy may comprise administering to the eye surface of the subject a pharmaceutical composition comprising an effective amount of at least one antiallergenic agent and a tear substitute in a pharmaceutically acceptable carrier.

The effective amount of antiallergenic agents in the formulation will depend on absorption, inactivation, and excretion rates of the drug as well as the delivery rate of the compound from the formulation. It is to be noted that dosage values may also vary with the severity of the condition to be alleviated. It is to be further understood that for any particular subject, specific dosage regimens should be adjusted over time according to the individual need and the professional judgment of the person administering or supervising the administration of the compositions. Typically, dosing will be determined using techniques known to one skilled in the art.

The dosage of any compound of the present invention will vary depending on the symptoms, age and other physical characteristics of the patient, the nature and severity of the disorder to be treated or prevented, the degree of comfort desired, the route of administration, and the form of the supplement. Any of the subject formulations may be administered in a single dose or in divided doses. Dosages for the formulations of the present invention may be readily determined by techniques known to those of skill in the art or as taught herein.

An effective dose or amount, and any possible effects on the timing of administration of the formulation, may need to be identified for any particular formulation of the present invention. This may be accomplished by routine experiment as described herein. The effectiveness of any formulation and method of treatment or prevention may be assessed by administering the formulation and assessing the effect of the administration by measuring one or more indices associated with the efficacy of the antiallergenic agent and with the degree of comfort to the patient, as described herein, and comparing the post-treatment values of these indices to the values of the same indices prior to treatment or by comparing the post-treatment values of these indices to the values of the same indices using a different formulation.

The precise time of administration and amount of any particular formulation that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

The combined use of several antiallergenic agents formulated into the compositions of the present invention may reduce the required dosage for any individual component because the onset and duration of effect of the different components may be complimentary. In such combined therapy, the different antiallergenic agents may be delivered together or separately, and simultaneously or at different times within the day.

6. Kits

In still another embodiment, this invention provides kits for the packaging and/or storage and/or use of the formulations described herein, as well as kits for the practice of the methods described herein. Thus, for example, kits may comprise one or more containers containing one or more ophthalmic solutions, tablets, or capsules of this invention. The kits can be designed to facilitate one or more aspects of shipping, use, and storage.

The kits may optionally include instructional materials containing directions (i.e., protocols) disclosing means of use of the formulations provided therein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g. CD ROM), and the like. Such media may include addresses to internet sites that provide such instructional materials.

All publications and patents mentioned herein are hereby incorporated by reference in their entirety as if each individual publication or patent was specifically and individually indicated to be incorporated by reference. In case of conflict, the present application, including any definitions herein, will control.

Exemplification

The invention now being generally described, it will be more readily understood by reference to the following examples, which are included merely for purposes of illustration of certain aspects and embodiments of the present invention, and are not intended to limit the invention.

### EXAMPLE 1

### Comfort Data for Three Concentrations of Ketotifen fumarate Compared to Zaditor® (Ketotifen Fumarate 0.025%)

Materials and Methods

The comfort of the current formulation of Zaditor® (ketotifen fumarate 0.025%) was compared with three concentrations of ketotifen fumarate (0.05%, 0.075% and 0.1%) in Genteal®. Subjects were asked to grade their baseline ocular discomfort on a scale of 0-4 (0=none, 1=intermittent awareness, 2=constant awareness, 3=intermittent discomfort, 4=constant discomfort). Forty microliters of Zaditor® (ketotifen fumarate 0.025%) was instilled in one eye while 40 microliters of ketotifen 0.05%, 0.075% or 0.1% in Genteal® was instilled in the contralateral eye. Subjects were then asked to grade the comfort of each eye immediately after instillation of the drop on a 0-8 scale (0 is most comfortable). After 1 minute (2 minutes, 5 minutes, and 10 minutes if necessary), subjects were asked to their grade ocular discomfort in both eyes on the 0-4 scale, blurring, lid caking and their eye drop preference. They were also asked to describe the sensation in their eyes.

Results

A) Comfort Data in Subjects Treated with Ketotifen Fumarate 0.025% and Ketotifen Fumarate 0.05% in Genteal®.

Four of the 6 subjects rated their baseline ocular discomfort as 0 on the 0-4 scale. One subject rated both eyes as 1 (intermittent awareness) and one subject rated his right eye 1 and his left eye 0. Upon instillation, the average drop comfort score for Zaditor® (ketotifen fumarate 0.025%) on the 0-8 scale was 2.3 (individual scores: 0, 0, 2, 4 [baseline 1], 2 [baseline 1], 6) while the average score for ketotifen 0.05% in Genteal® was 1.7 (individual scores: 0, 0, 1, 2, 3 [baseline 1], 4). Adjectives used to describe the sensation in the eye after instillation of ketotifen 0.05% in Genteal® included “wet” and “cool”. Adjectives used to describe the ketotifen 0.025% included “dry”, “cool” and “wet”.

Adverse events: One person experienced blurring and stinging in both eyes immediately after instillation (less in the eye receiving the ketotifen 0.05% in Genteal®), while another subject reported blurring in the eye receiving Zaditor® (ketotifen fumarate 0.025%) (this subject reported a 1 in baseline ocular discomfort in the eye that was blurry). One subject reported lid caking in the eye that received ketotifen 0.05% and Genteal®.

One minute after drop instillation, three subjects described the eye receiving ketotifen 0.05% in Genteal® as feeling wet. After 1 minute, all subjects reported that they preferred the ketotifen fumarate 0.05% in Genteal® eye drop over Zaditor® (0.025% ketotifen fumarate).

B) Comfort Data in Subjects Treated with Ketotifen Fumarate 0.025% and Ketotifen Fumarate 0.075% in Genteal®.

Five subjects rated their baseline ocular discomfort as 0 on the 0-4 scale. One subject rated the right eye 1 (intermittent awareness) and the left eye 0. Upon instillation, the average drop comfort score for Zaditor® (ketotifen fumarate 0.025%) on the 0-8 scale, where 0 is most comfortable, was 3.0 (individual scores: 2, 2, 3, 3, 3, 5) while the average score for ketotifen 0.075% in Genteal® was 3.3 (individual scores: 2, 2, 3, 4, 4, 5).

Three subjects reported that the Zaditor® (ketotifen fumarate 0.025%) was more comfortable than the 0.075% ketotifen in Genteal® upon instillation while 3 reported that the two were equally comfortable.

Adverse events: One subject reported blurring for 10 sec after instillation of 0.025% and 20 seconds after instillation of 0.075% while another subject experienced blurring in the eye that received 0.075% 1 minute and 2 minutes after drop instillation.

One minute after drug instillation, four subjects reported that they preferred ketotifen 0.025%, one subject preferred 0.075% ketotifen in Genteal® and one subject had no preference. Two minutes after instillation, 2 subjects reported ocular awareness in both eyes but preferred ketotifen 0.025% and one subject reported awareness in the eye receiving ketotifen 0.025% and preferred ketotifen 0.075%.

C) Comfort Data in Subjects Treated with Ketotifen Fumarate 0.025% and Ketotifen Fumarate 0.1% in Genteal®.

Four of the 6 subjects rated their baseline ocular discomfort as 0 on the 0-4 scale and two gave both eyes a rating of 1. Upon instillation, the average drop comfort score for Zaditor® (ketotifen fumarate 0.025% on the 0-8 scale, where 0 is most comfortable, was 1.7 (individual scores: 0.25, 0, 1, 3, 2, 4) while the average score for ketotifen 0.1% in Genteal® was 3.1 (individual scores: 0.5, 1, 2, 4, 5, 6). Of the subjects that had a baseline score of 1, one subject rated discomfort upon instillation 3 and 4 for ketotifen 0.025% and 0.075 in Genteal® respectively and the other subject gave ratings of 0 and 1. All subjects reported that the Zaditor® (ketotifen fumarate 0.025%) was more comfortable upon instillation.

Adverse events: One subject reported blurring at 1 after drop instillation and blurring in the eye receiving ketotifen 0.1% in Genteal® at 5 minutes after drop instillation. One subject reported that the stinging that occurred in both eyes upon instillation lasted slightly longer in the eye that received ketotifen 0.1% in Genteal®. One of the two subjects that reported a score of 1 on baseline ocular discomfort recorded constant discomfort in both eyes for two minutes, intermittent discomfort in the eye receiving ketotifen 0.1% in Genteal® from minute 2-3 and intermittent awareness of the eye that received ketotifen 0.1% in Genteal® up to 20 minutes after drop instillation. Two subjects reported discomfort for one minute in the eye receiving ketotifen 0.1% in Genteal® and one of the two reported awareness in the eye that received ketotifen 0.1% for 5 minutes. The other three subjects reported no discomfort after 15 seconds.

Adjectives used to describe the ketotifen 0.1% in Genteal included “irritating”, “cool”, “refreshing” and “burning”. One minute after drop instillation, 4 subjects reported that they preferred the current Zaditor® (ketotifen fumarate 0.025%) over the ketotifen 0.1% in Genteal® while 2 subjects did not report a preference.

### EXAMPLE 2

### Randomized Placebo-Controlled Evaluation of the Duration of Action of 0.05% Ketotifen Fumarate and 0.05% Ketotifen Fumarate in Genteal® in the Conjunctival Allergen Challenge (CAC) Model

Materials and Methods

The following studies were performed with healthy adult volunteers with known histories of allergic conjunctivitis or rhinoconjunctivitis. These volunteers have historically manifested a positive conjunctival allergen challenge reaction and additionally meet all entry criteria.

At the initial visit (baseline screening (Visit 1, Day 0), subjects' informed consent were obtained and demographic data, medical and medication history, and visual acuity was recorded. A urine pregnancy test was given to all women of child bearing potential.

A slit-lamp exam was performed to exclude subjects with disallowed ocular conditions, or those who were currently exhibiting signs and symptoms of allergic conjunctivitis (defined as >1+redness in any vessel bed or the presence of any itching in either eye). Each subject was randomized to receive ketotifen fumarate 0.05% in Genteal® Lubricant Eyedrop in one eye and Placebo Genteal® Eyedrop in the contralateral eye.

Visual acuity was recorded and each subject was instructed to return for the second part of the visit in 15.5 hours. When the subject returned for the second part of the visit, subjects were queried regarding adverse events and a slit lamp examination was performed. A conjunctival allergen challenge (CAC) was performed bilaterally with cat hair/dander or trees (serially diluted in a buffered saline and administered via a micropipette).

One drop of solubilized allergen at the lowest concentration was instilled into the conjunctival cul-de-sac bilaterally. If the subject failed to react within 10 minutes, increasing doses were instilled at a minimum of ten minute intervals until a positive reaction was elicited. A positive CAC was defined as >2.0+itching and >2.0+hyperemia in two of the three vessel beds bilaterally within 10 minutes of receiving that dose of allergen. The technician instilling the allergen initiated the titration from the lowest concentration. Itching, tearing and lid swelling was graded by the subject and hyperemia and chemosis was graded by the investigator after each 10 minute interval. When a positive CAC was elicited in either eye, that eye was no longer challenged with antigen. The contralateral eye was challenged until a positive CAC was elicited in that eye or until the subject experienced tickling/swelling of the palate, which could jeopardize the safety of the subject. Any subject who failed to test positively in either eye was excluded from the study.

The grading of itching, tearing and lid swelling was done subjectively using a standardized scale.

Results

No adverse events were reported after study drug instillation.

Three subjects (subjects 2, 4, and 17) failed to reach a positive CAC reaction in one eye. In all three cases, itching scores in the “CAC non-positive” eye had started to decrease despite continued increase in redness. The eyes in question reached positive redness scores (>+redness in at least two vessel beds) at the final administered dose despite a final itching score of less than 2.

When analyzing the “allergen dose required to elicit a positive conjunctival allergen challenge,” data from these subjects was not used since a true positive CAC challenge was never attained. However, for completeness, all data analyses was performed by excluding subjects 2, 4, and 17 or by including them (‘intent to treat’).

Primary Efficacy Analysis

The allergen dose required to elicit a positive conjunctival allergen challenge at Visit 1 was analyzed.

TABLE 1
A Primary Efficacy Analysis excluding subjects 2, 4, and 17 per protocol
deviations described above:
Mean dose of allergen required to elicit positive CAC reaction

| | |
|---|---|
| Ketotifen 0.05% in Genteal ® | 397 AU |
| Genteal ® | 264 AU |

p = 0.03126
n = 13

TABLE 2
A Primary Efficacy Analysis including subjects 2, 4, and 17 (intent to
treat).
Mean dose of allergen required to elicit positive CAC reaction

| | |
|---|---|
| Ketotifen 0.05% in Genteal ® | 349 AU |
| Genteal ® | 256 AU |

p = 0.12500
n = 16

Secondary Efficacy Analysis

The secondary efficacy variables were mean itching and redness at 10 minute intervals following allergen challenge.

TABLE 3
A Secondary Efficacy Analysis excluding subjects 2, 4, and 17 per
protocol deviations described above:

| | Treatment | | |
|---|---|---|---|
| | | Ketotifen 0.05% | |
| | Genteal ® | in Genteal ® | p-value |
| Mean Itching Score: | 0.949 | 0.684 | 0.0037 |
| Mean Ciliary Redness Score: | 0.912 | 0.831 | 0.4016 |
| Mean Conj Redness Score: | 0.963 | 0.824 | 0.2062 |
| Mean Epi Redness Score: | 0.515 | 0.375 | 0.0194 |
| Mean Chemosis Score: | 0.0441 | 0.0294 | 0.7500 |

TABLE 4
A Secondary Efficacy Analysis including subjects 2, 4, and 17
(intent to treat).

| | Treatment | | |
|---|---|---|---|
| | | Ketotifen 0.05% | |
| | Genteal ® | in Genteal ® | p-value |
| Mean Itching Score: | 0.963 | 0.685 | 0.0038 |
| Mean Ciliary Redness Score: | 0.944 | 0.883 | 0.5700 |
| Mean Conj Redness Score: | 0.951 | 0.883 | 0.6960 |
| Mean Epi Redness Score: | 0.599 | 0.451 | 0.0369 |
| Mean Chemosis Score: | 0.0556 | 0.0494 | 1.0000 |

When subjects 2, 4 and 17 were excluded for deviation from protocol, the average dose of antigen required to elicit a positive CAC reaction was significantly greater in the eyes that received ketotifen 0.05% in Genteal®D compared to the eyes that received Genteal® alone, suggesting that the drug provided some protection against the signs and symptoms of allergic conjunctivitis.

When determining the mean itching and redness scores for a subject, any data from a timepoint at which only one eye was challenged with a higher dose of allergen were excluded. When the mean itching and redness scores for all eyes treated with ketotifen 0.05% in Genteal® were averaged and compared with the average itching and redness scores for eyes treated with Genteal® alone, itching and episcleral (epi) redness were significantly decreased in those eyes that received the active treatment.

A statistically significant difference in the antigen dose required to elicit an allergic reaction and more specifically a difference in mean itching and episcleral (epi) redness showed that 0.5% Ketotifen fumarate formulated with Genteal® attenuated the ocular allergic response 16 hours after administration.

TABLE 5
A comparison of Zaditor (ketotifen fumarate 0.025%) with ketotifen 0.05% in
Genteal ® in six subjects

| | | | Drop | | | | | | | | |
|---|---|---|---|---|---|---|---|---|---|---|---|
| | | | Comfort | | | | | | | | |
| | | | Upon | Ocular Discomfort | | | | | | | |
| | | | Instillation | 0-4 Scale | | | | | | | |
| Comparison | Patient | Drug | (0-8) | Pre | 1 min | 2 min | 5 min | Blurring | Lid Caking | Preferred Drop | Comments |
| Zaditor (A) | 1 | A | 0 | 0 | 0 | 0 | 0 | N | N | B | |
| vs. | | B | 0 | 0 | 0 | 0 | 0 | N | N | | |
| ketotifen | 2 | A | 6 | 0 | 3 | 0 | 0 | Y | N | B | stinging OU |
| 0.05% in | | B | 4 | 0 | 1 | 0 | 0 | Y | N | | |
| Genteal (B) | 3 | A | 2 | 0 | 0 | 0 | 0 | N | N | B | stickier, cool |
| | | | | | | | | | | | feeling at 2 |
| | | | | | | | | | | | minutes |
| | | B | 3 | 1 | 0 | 0 | 0 | N | N | | cool feeling |
| | | | | | | | | | | | at 2 minutes |
| | 4 | A | 0 | 0 | 0 | 0 | 0 | N | N | B | dry feeling |
| | | B | 0 | 0 | 0 | 0 | 0 | N | N | | wet feeling |
| | 5 | A | 4 | 0 | 0 | 0 | 0 | N | N | B | blurring |
| | | | | | | | | | | | upon |
| | | | | | | | | | | | instillation, |
| | | | | | | | | | | | cool |
| | | B | 2 | 0 | 0 | 0 | 0 | N | N | | wet feeling |
| | 6 | A | 0 | 2 | 0 | 0 | 0 | N | N | B | wet feeling |
| | | B | 0 | 1 | 0 | 0 | 0 | N | N | | OU |

| | | |
|---|---|---|
| Averages | A | 2.3 |
| | B | 1.7 |

TABLE 6
A comparison of Zaditor (ketotifen fumarate 0.025%) with ketotifen 0.075% in
Genteal ® in six subjects

| | | | Drop | | | | | | | | |
|---|---|---|---|---|---|---|---|---|---|---|---|
| | | | Comfort | | | | | | | | |
| | | | Upon | Ocular Discomfort | | | | | | | |
| | | | Instillation | 0-4 Scale | | | | | | | |
| Comparison | Patient | Drug | (0-8) | Pre | 1 min | 2 min | 5 min | Blurring | Lid Caking | Preferred Drop | Comments |
| Zaditor (A) | 1 | A | 3 | 0 | 0 | 0 | 0 | N | N | A | |
| vs. | | B | 4 | 0 | 0 | 0 | 0 | N | N | | soothing |
| ketotifen | 2 | A | 1 | 0 | 1 | 1 | 0 | N | N | A | |
| 0.075% in | | B | 3 | 0 | 1 | 1 | 0 | Y | N | | |
| Genteal (B) | 3 | A | 2 | 0 | 0 | 0 | 0 | N | N | No | |
| | | B | 2 | 1 | 1 | 0 | 0 | N | N | | |
| | 4 | A | 2 | 0 | 1 | 1 | 0 | N | N | B | |
| | | B | 2 | 0 | 0 | 0 | 0 | N | N | | |
| | 5 | A | 5 | 0 | 2 | 1 | 0 | Y* | N | A | *blurring for |
| | | | | | | | | | | | 10 sec upon |
| | | | | | | | | | | | instillation |
| | | B | 5 | 0 | 2 | 1 | 0 | Y* | N | | *blurring for |
| | | | | | | | | | | | 20 sec upon |
| | | | | | | | | | | | instillation |
| | 6 | A | 3 | 0 | 0 | 0 | 0 | N | N | A | |
| | | B | 4 | 0 | 1 | 0 | 0 | N | N | | |

| | | |
|---|---|---|
| Averages | A | 3 |
| | B | 3.3 |

TABLE 7
A comparison of Zaditor (ketotifen fumarate 0.025%) with ketotifen 0.01%
in Genteal ® in six subjects

| | | | Drop | | | | | | | | |
|---|---|---|---|---|---|---|---|---|---|---|---|
| | | | Comfort | | | | | | | | |
| | | | Upon | Ocular Discomfort | | | | | | | |
| | | | Instillation | 0-4 Scale | | | | | | | |
| Comparison | Patient | Drug | (0-8) | Pre | 1 min | 2 min | 5 min | Blurring | Lid Caking | Preferred Drop | Comments |
| Zaditor(A) | 1 | A | 4 | 0 | 0 | 0 | 0 | N | N | A | |
| vs. | | B | 6 | 0 | 4 | 0 | 0 | N | N | | after initial |
| ketotifen | | | | | | | | | | | sting, felt |
| 0.1% in | | | | | | | | | | | soothing, |
| Genteal (B) | | | | | | | | | | | refreshing |
| | 2 | A | 2 | 0 | 0 | 0 | 0 | N | N | A | irritating |
| | | B | 5 | 0 | 2 | 1 | 1 | N | N | | |
| | 3 | A | 3 | 1 | 4 | 1 | 0 | Y | N | A | |
| | | B | 4 | 1 | 4 | 3 | 1 | Y | Y | | cool, |
| | | | | | | | | | | | burning; |
| | | | | | | | | | | | intermittent |
| | | | | | | | | | | | awareness |
| | | | | | | | | | | | for 20 min |
| | 4 | A | 1 | 0 | 0 | 0 | 0 | N | N | A | |
| | | B | 2 | 0 | 0 | 0 | 0 | N | N | | stinging was |
| | | | | | | | | | | | a little |
| | | | | | | | | | | | stronger, |
| | | | | | | | | | | | lasted longer |
| | | | | | | | | | | | than other |
| | | | | | | | | | | | eye |
| | 5 | A | 0 | 1 | 0 | 0 | 0 | N | N | No | No |
| | | | | | | | | | | | discomfort |
| | | | | | | | | | | | after 5 sec |
| | | B | 1 | 1 | 0 | 0 | 0 | N | N | | No |
| | | | | | | | | | | | discomfort |
| | | | | | | | | | | | after 5 sec |
| | 6 | A | 0.25 | 0 | 0 | 0 | 0 | N | N | No | no |
| | | | | | | | | | | | discomfort |
| | | | | | | | | | | | after 15 sec |
| | | B | 0.5 | 0 | 0 | 0 | 0 | N | N | | no |
| | | | | | | | | | | | discomfort |
| | | | | | | | | | | | after 15 sec |
| Averages | | A | 1.7 | | | | | | | | |
| | | B | 3.1 | | | | | | | | |

### EQUIVALENTS

Those skilled in the art will recognize, or be able to ascertain using no more than routine experimentation, many equivalents to the specific embodiments of the invention described herein. Such equivalents are intended to be encompassed by the following claims. claims:

1. A pharmaceutical composition comprising an effective amount of at least one antiallergenic agent and a tear substitute in a pharmaceutically acceptable carrier.

2. The pharmaceutical composition of claim 1, wherein the antiallergenic agent is selected from the group consisting of: an antihistamine, a mast cell stabilizer, a drug with multiple modes of action and a NSAID.

3. The pharmaceutical composition of claim 2, wherein the antiallergenic agent is a drug with multiple modes of action.

4. The pharmaceutical composition of claim 3, wherein the drug with multiple modes of action is selected from the group consisting of: azelastine, epinastine and ketotifen fumarate.

5. The pharmaceutical composition of claim 4, wherein the drug with multiple modes of action is ketotifen fumarate.

6. The pharmaceutical composition of claim 5, wherein the effective amount of ketotifen fumarate is between about 0.025% and about 0.1%.

7. The pharmaceutical composition of claim 6, wherein the effective amount of ketotifen fumarate is between about 0.05% and about 0.075%.

8. The pharmaceutical composition of claim 5, wherein the tear substitute contains hydroxypropylmethylcellulose.

9. The pharmaceutical composition of claim 8, wherein the tear substitute is Genteal®.

10. The pharmaceutical composition of claim 6, wherein the tear substitute contains hydroxypropylmethylcellulose.

11. The pharmaceutical composition of claim 1, wherein the composition is formulated for topical ophthalmic use.

12. The pharmaceutical composition of claim 1, wherein the composition comprises at least two antiallergenic agents and a tear substitute.

13. The pharmaceutical composition of claim 1, further comprising an effective amount of a vasoconstrictor.

14. A method of treating ocular allergy in a subject comprising: administering to the eye surface of the subject a pharmaceutical composition comprising an effective amount of at least one antiallergenic agent and a tear substitute in a pharmaceutically acceptable carrier.

15. The method of claim 14, wherein the antiallergenic agent is selected from the group consisting of: an antihistamine, a mast cell stabilizer, a drug with multiple modes of action and a NSAID.

16. The method of claim 15, wherein the antiallergenic agent is a drug with multiple modes of action.

17. The method of claim 16, wherein the drug with multiple modes of action is ketotifen fumarate.

18. The method of claim 17, wherein the effective amount of ketotifen fumarate is between about 0.025% and about 0.1% and the tear substitute contains hydroxypropylmethylcellulose.

19. A kit comprising a pharmaceutical composition comprising an effective amount of at least one antiallergenic agent and a tear substitute in a pharmaceutically acceptable carrier.

20. The kit of claim 19, further comprising instructions for use.
